(19) United States
(12) Patent Application Publication
(10) Pub. No.: US 2009/0012544 A1
(43) Pub. Date: Jan. 8, 2009

(54) GASTROINTESTINAL BYPASS SLEEVE AS AN ADJUNCT TO BARIATRIC SURGERY

(75) Inventors: Chris Thompson, Boston, MA (US); Mitchell Roslin, New York, NY (US); Mitchell Dann, Wilson, WY (US); Greg Fluet, Jackson, WY (US); James Wright, Carpinteria, CA (US)

Correspondence Address: KNOBBE MARTENS OLSON & BEAR LLP, 2040 MAIN STREET, FOURTEENTH FLOOR, IRVINE, CA, 92614, US

(73) Assignee: VALEN TX, INC., Carpinteria, CA (US)

(21) Appl. No.: 12/135,989

(22) Filed: Jun. 9, 2008

Related U.S. Application Data
(60) Provisional application No. 60/943,014, filed on Jun. 8, 2007.
(60) Provisional application No. 60/982,692, filed on Oct. 25, 2007.

(51) Int. Cl. A61B 17/11

(52) U.S. Cl. 606/156

(57) ABSTRACT

Disclosed herein are systems and methods for treating a patient that has undergone a bariatric surgical procedure, to promote weight loss in the patient. The systems and methods can involve positioning a gastrointestinal bypass sleeve within a portion of the altered GI anatomy to create or restore a restriction, and/or create a malabsorptive effect via a gastric and partial intestinal bypass. The bypass sleeve can include a proximal attachment element for attaching the proximal end of the sleeve in a penetrating or non-penetrating manner at the gastroesophageal junction, stomach, neo-stomach, or other locations. The bypass can be placed during the same operative session as the bariatric surgical procedure, or alternatively at a later date.

### PRIORITY CLAIM

The present application claims priority under 35 U.S.C. § 119(e) to U.S. Provisional Application No. 60/943,014, filed Jun. 8, 2007, and U.S. Provisional Application No. 60/982,692 filed Nov. 25, 2007, both of which are hereby incorporated by reference in their entireties.

### APPLICATIONS INCORPORATED BY REFERENCE

Various features of, for example, gastrointestinal bypass sleeves, attachment cuffs, and/or toposcopic delivery methods that can be used or adapted for use with systems and methods disclosed herein can be found, for example, at U.S. patent application Ser. No. 10/698,148, filed Oct. 31, 2003, published May 13, 2004 as U.S. Patent Pub. No. 2004-0092892 A1 and entitled “APPARATUS AND METHODS FOR TREATMENT OF MORBID OBESITY” (and may be referred to herein as the “Kagan '148 application or Kagan '892 publication”); U.S. patent application Ser. No. 11/025,364, filed Dec. 29, 2004, published Aug. 11, 2005 as U.S. Patent Pub. No. 2005-0177181 A1 and entitled “DEVICES AND METHODS FOR TREATING MORBID OBESITY” (and may be referred to herein as the “Kagan '181 publication”); U.S. patent application Ser. No. 11/124,634, filed May 5, 2005, published Jan. 26, 2006 as U.S. Patent Pub. No. 2006-0020247 A1 and entitled “DEVICES AND METHODS FOR ATTACHMENT OF AN ENDOLUMENAL GASTROINTESTINAL IMPLANT” (and may be referred to herein as the “Kagan '247 publication”); U.S. patent application Ser. No. 11/400,724, filed Apr. 7, 2006, published Jan. 11, 2007 as U.S. Patent Pub. No. 2007-0010794 A1 and entitled “DEVICES AND METHODS FOR ENDOLUMENAL GASTROINTESTINAL BYPASS” (and may be referred to herein as the “Dann '794 publication”); and U.S. patent application Ser. No. 11/548,605, filed Oct. 11, 2006, published Aug. 23, 2007 as U.S. Pub. No. 2007-0198074 A1 and entitled “DEVICES AND METHODS FOR ENDOLUMENAL GASTROINTESTINAL BYPASS” (and may be referred to herein as the “Dann '605 application” or “Dann '074 publication”); and U.S. Provisional Application No. 60/943,014 filed Jun. 8, 2007 and entitled “GASTROINTESTINAL BYPASS SLEEVE AS AN ADJUNCT TO BARIATRIC SURGERY” are hereby incorporated by reference in their entireties herein, as well as any additional applications, patents, or publications noted in the specification below.

### SUMMARY OF THE INVENTION

Disclosed herein are systems and method for treating a patient. In one embodiment, disclosed is a method of treating a patient, including the steps of identifying a patient who has undergone a bariatric surgical procedure; and positioning a gastrointestinal sleeve device with a proximal end, distal end, and elongate body in a patient such that the proximal end of the sleeve device is positioned in the stomach and the distal end of the sleeve device is positioned in the intestine. The proximal end of the sleeve can be positioned in the native stomach or a neo-stomach, in some embodiments. The bariatric surgical procedure can be, for example, a Roux-en-Y gastric bypass, sleeve gastrectomy, sleeve gastrectomy with duodenal switch, biliopancreatic diversion, biliopancreatic diversion with duodenal switch, vertical banded gastroplasty, vertical banded gastroplasty with gastric bypass, Lap-Band procedure, or Magenstrasse and Mill procedure. In some embodiments, the method further includes the step of securing the proximal end of the gastrointestinal sleeve with respect to the neo-stomach. In some embodiments, the gastrointestinal sleeve device is operably attached to a proximal attachment element at the proximal end of the sleeve device. Securing the proximal end of the gastrointestinal sleeve can involve either penetratingly or nonpenetratingly attaching the sleeve device through a wall of the neo-stomach. The proximal attachment element could include any number of features, for example, an attachment cuff, an expandable dome, an outwardly-biased funnel, a stent, an expandable structure, a balloon, or woven shape memory material in some embodiments. In some embodiments, the step of positioning the distal end of the sleeve device within the intestine involves. toposcopically everting the sleeve within the intestine. In some embodiments, the method includes the step of leaving the gastrointestinal sleeve device within the patient for at least about 2 weeks. In some embodiments, positioning the distal end of the sleeve device within the intestine involves positioning the distal end of the sleeve device within the duodenum, jejunum, or the ileum. Positioning the sleeve device can occur in the same operative session as the bariatric surgical procedure, or an operative session at a later date.

In some embodiments, disclosed herein is a method of treating a patient, including the steps of identifying a patient who has undergone a bariatric surgical procedure; and positioning a gastrointestinal sleeve device with a proximal end, distal end, and elongate body in a patient such that the proximal end of the sleeve device is positioned in the stomach and the distal end of the sleeve device is positioned in the intestine.

In some embodiments, disclosed is a method of treating a patient, including the steps of identifying a patient who has undergone a bariatric surgical procedure; and positioning a gastrointestinal sleeve device in a patient such that the proximal end of the sleeve device is positioned at the gastroesophageal junction and the distal end of the sleeve device is positioned in the intestine.

In some embodiments, disclosed is a method of treating a patient, including the steps of identifying a patient who has undergone a bariatric surgical procedure; and positioning a gastrointestinal sleeve device in a patient such that the proximal end of the sleeve device is positioned at the gastroesophageal junction and the distal end of the sleeve device is positioned in the stomach to create a restrictive effect.

In some embodiments, disclosed is a method of treating a patient, including the steps of providing a gastrointestinal sleeve device, the sleeve device comprising one or more fins configured to radially expand from the sleeve device against the greater curvature of the stomach; positioning the gastrointestinal sleeve device in the patient such that the fins radially expand against the greater curvature of the stomach; and stapling the stomach to perform a sleeve gastrectomy such that the fins are captured in the resulting staple line, securing the fins within the neo-stomach created by the sleeve gastrectomy.

### BRIEF DESCRIPTION OF THE DRAWINGS

FIG. 1 schematically illustrates normal stomach anatomy.

FIG. 2 schematically illustrates anatomy following vertical banded gastroplasty.

FIG. 3 schematically illustrates anatomy following sleeve gastrectomy.

FIG. 4 schematically illustrates anatomy following a sleeve gastrectomy and a duodenal switch.

FIG. 5A schematically illustrates anatomy following a Magenstrasse and Mill procedure, an unbanded long lesser curve gastroplasty without resection.

FIG. 5B schematically illustrates anatomy following a sleeve gastrectomy and biliopancreatic diversion without duodenal switch (also known as the Scopinaro procedure).

FIG. 6 schematically illustrates GI anatomy status-post Roux-en-Y bypass.

FIG. 7 schematically illustrates an endoscopic bypass sleeve implanted in the proximal neostomach attached transmurally via a cuff and transmural T-tags in a patient status-post Roux-en-Y bypass, according to one embodiment of the invention.

FIG. 8 schematically shows an endoscopic bypass sleeve implanted in the proximal neostomach attached via staples or sutures in a patient status-post Roux-en-Y bypass, according to one embodiment of the invention.

FIG. 9 schematically shows an endoscopic bypass sleeve implanted in the proximal neostomach attached via an outwardly biased funnel in a patient status-post Roux-en-Y bypass, according to one embodiment of the invention.

FIG. 10 schematically shows an endoscopic bypass sleeve implanted in the proximal neostomach secured via an expandable stent in a patient status-post Roux-en-Y bypass, according to one embodiment of the invention.

FIG. 11 schematically shows an endoscopic bypass sleeve implanted in the proximal neostomach secured via an inflatable or fillable toroidal ring or balloon in a patient status-post Roux-en-Y bypass, according to one embodiment of the invention.

FIG. 12 schematically shows an endoscopic bypass sleeve implanted in the proximal neostomach secured via an expandable dome-shaped element in a patient status-post Roux-en-Y bypass, according to one embodiment of the invention.

FIG. 13 schematically shows an endoscopic bypass sleeve implanted in the proximal neostomach secured more proximally in the vicinity of the gastroesophageal junction in a patient status-post Roux-en-Y bypass, according to one embodiment of the invention.

FIG. 14 schematically shows an endoscopic bypass sleeve implanted in the proximal neostomach with an expandable element around the portion of the sleeve in the neostomach for holding the sleeve in place, in a patient status-post Roux-en-Y bypass, according to one embodiment of the invention.

FIGS. 15A-B schematically shows an endoscopic bypass sleeve implanted in the proximal neostomach with an intragastric support system for holding the sleeve in place, in a patient status-post Roux-en-Y bypass, according to some embodiments of the invention.

FIG. 16 schematically shows an endoscopic bypass sleeve implanted in the proximal neostomach with an expandable woven shape memory material portion within the neostomach for holding the sleeve in place, in a patient status-post Roux-en-Y bypass, according to one embodiment of the invention.

FIGS. 17A-B schematically illustrate an endoscopic bypass sleeve with an outwardly biased portion and a neck portion configured to operably engage the proximal Roux-en-Y anastomosis, according to some embodiments of the invention.

FIGS. 18-20B schematically illustrate a method of delivering an endoscopic bypass sleeve toposcopically, according to one embodiment of the invention.

FIG. 21 schematically illustrates anatomy following a sleeve gastrectomy procedure, also showing the intestines.

FIG. 22 schematically illustrates a bypass sleeve with its distal end positioned in the ileum to functionally recreate the results of a duodenal switch procedure, according to one embodiment of the invention.

FIGS. 23A-B schematically illustrate a bypass sleeve with one or more radial fins to configured to secure the sleeve to a neo-stomach staple line during a sleeve gastrectomy procedure, according to one embodiment of the invention.

### DETAILED DESCRIPTION OF THE PREFERRED EMBODIMENT

Bariatric surgery procedures to induce weight loss, such as in morbidly obese patients for example, include, for example, the Roux-en-Y gastric bypass, sleeve gastrectomy (SG), sleeve gastrectomy with duodenal switch (SG-DS) or biliopancreatic diversion (BPD), vertical banded gastroplasty (VBG), VBG with bypass (VBG-GB), Magenstrasse and Mill procedure, and adjustable gastric banding (AGB). Bariatric surgical procedures generally seek to promote weight loss through at least one of two mechanisms: (1) creating a restrictive effect (e.g., by reducing the effective volume of the stomach) and/or (2) creating a malabsorptive effect (e.g., by creating an intestinal bypass).

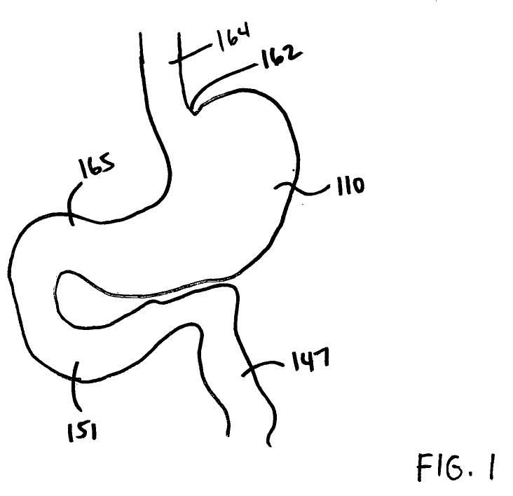

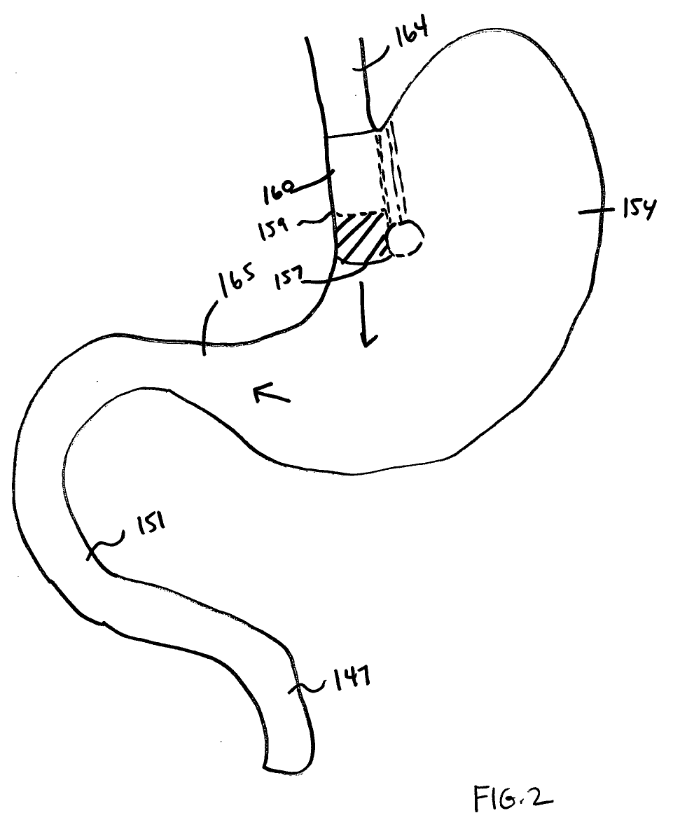

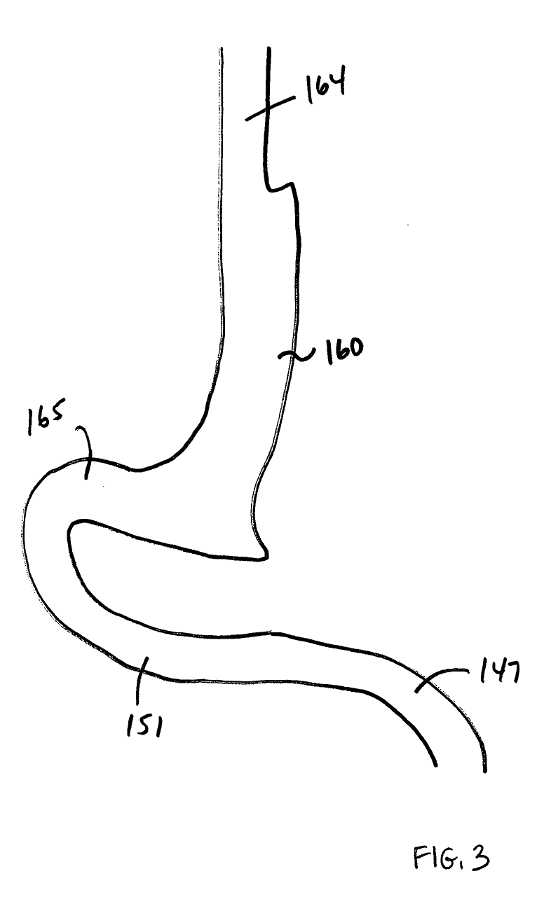

The normal native stomach anatomy is shown in FIG. 1, illustrating the esophagus 164, gastroesophageal junction 162, stomach 110, pylorus 165, duodenum 151, and jejunum 147. Bariatric surgical procedures such that those described above, with the exception of gastric banding, permanently alter the gastrointestinal anatomy of a patient, as shown in FIGS. 2-5B and described briefly below.

FIG. 2 illustrates anatomy status-post a vertical banded gastroplasty. As shown, a large portion of the stomach has been divided and stapled, leaving a small neo-stomach 160 surrounded by one or more restrictive bands 159. A small aperture 157 formed in the distal end of the neo-stomach 160 can serve as a proximal anastomotic conduit 152 to the bypassed stomach 154 as shown.

FIG. 3 illustrates anatomy status-post a sleeve gastrectomy procedure, where the stomach is significantly reduced in size via surgical removal of a large portion of the stomach following the greater curve. The open remaining edges are then sutured or stapled together to form a sleeve or tubular neo-stomach 160 as shown.

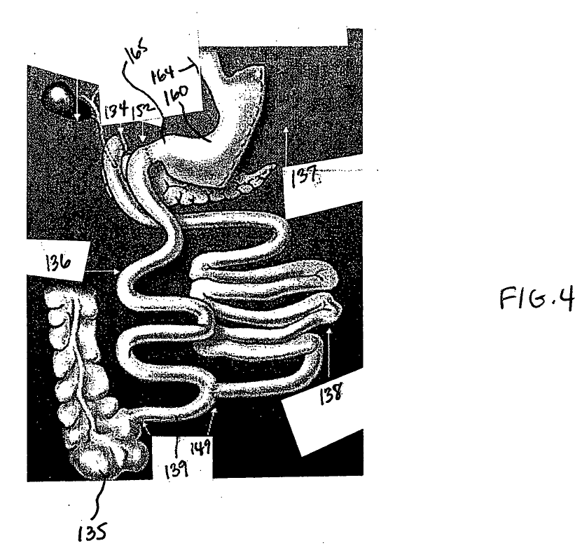

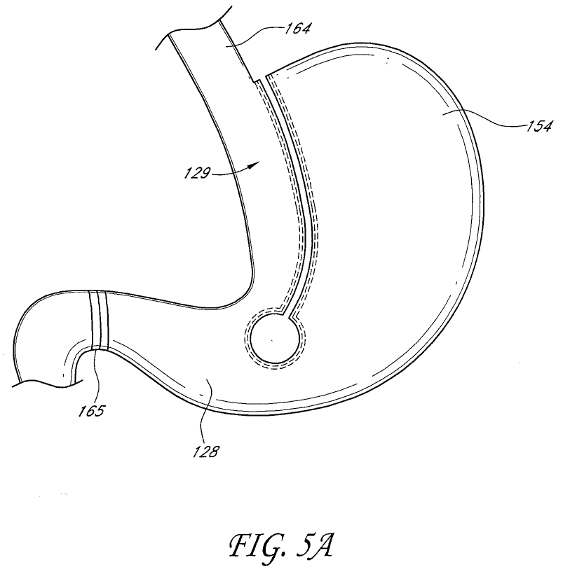

FIG. 4 illustrates anatomy status-post a duodenal switch procedure, where part of the stomach 137 is resected, leaving a smaller volume neo-stomach 160. The duodenum is ligated just distal to the pylorus 165 (leaving the pyloric valve intact), and the proximal end 134 closed to form a bilio-pancreatic loop 138. The gallbladder can also be removed. A portion of the distal ileum 136 is proximally ligated and then anastomosed proximally 152 just distal to the pylorus 165, which is preserved with this procedure, forming a digestive loop 136. The distal end of the duodenal-ileal bilio-pancreatic loop can then be anastomosed to the digestive loop as shown to form distal anastomosis 149. A short loop of ileum about 75-100 cm serves as a common pathway 139 proximal to the ileocecal valve and large intestine 135.

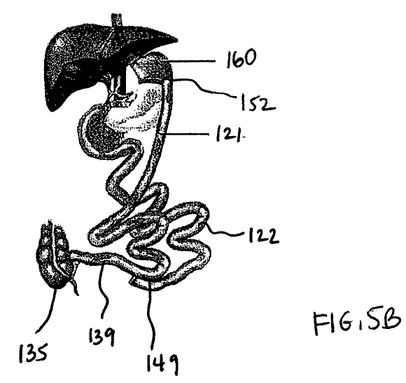

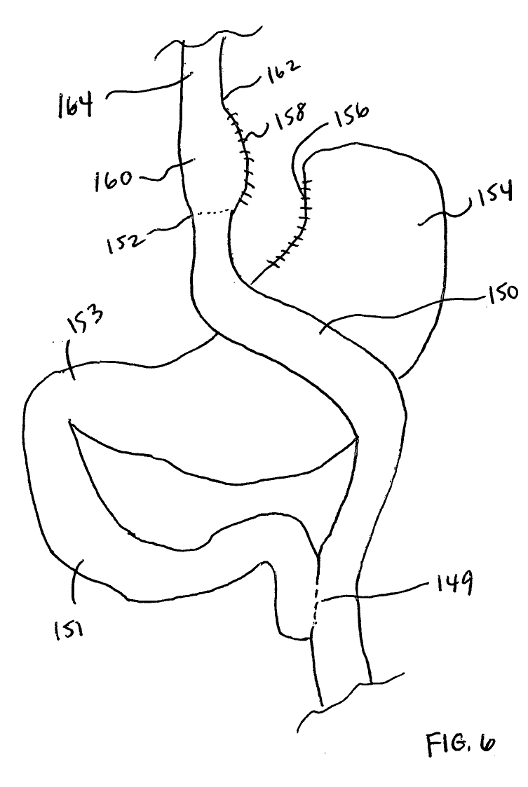

FIG. 5A illustrates anatomy status-post a Magenstrasse and Mill procedure, a form of gastroplasty where a portion of the stomach near the lesser curve is fashioned into a long and narrow tube 129 (the “Magenstrasse”) which conveys food from the esophagus 164 to the antral Mill 128, excluding a large portion 154 of the stomach. Pyloric 165 function is preserved with this procedure.

FIG. 5B illustrates anatomy status-post a biliopancreatic diversion (Scopinaro procedure) where a large portion of the stomach, including the pylorus, has been removed. The duodenum is then ligated, forming a biliopancreatic loop 122. The remaining neo-stomach 160 is then anastomosed to a loop of ileum that has been proximally ligated, forming a digestive loop 121. The distal end of the biliopancreatic loop 122 is then anastomosed distally at 149 to a portion of the digestive loop 121. A short loop of ileum serves as a common pathway 139 proximal to the ileocecal valve and large intestine 135 as shown.

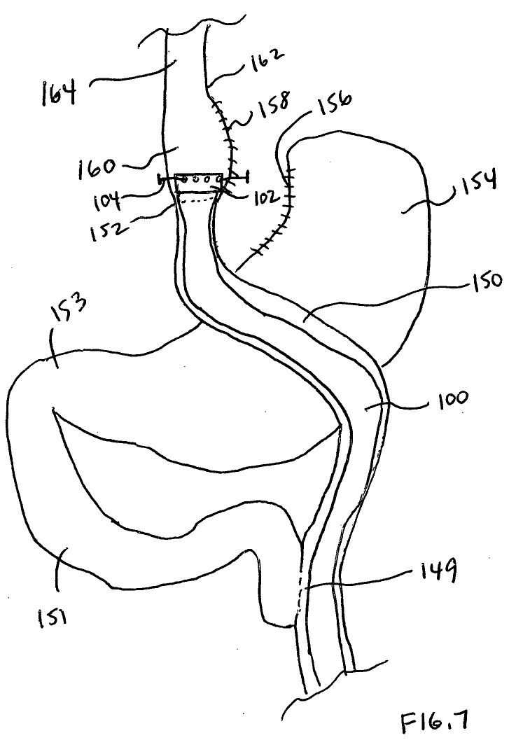

FIG. 6 illustrates anatomy status-post Roux-en-Y bypass. Illustrated is the esophagus 164, gastroesophageal junction (GEJ) 162, neo-stomach 160, neo-stomach staple or suture lines 158, bypassed stomach staple or suture lines 156, bypassed stomach 154 and pylorus 153, duodenum 151, proximal neo-stomach-jejunal anastomosis 152, Roux intestinal limb 150, and distal intestinal anastomosis 149.

A common result of all these bariatric surgical procedures is that they leave a residual stomach or “neo-stomach” that is a remnant portion of the natural stomach. The size of the neo-stomach varies by procedure and physician, but can be no more than about 90%, 80%, 70%, 60%, 50%, 40%, 30%, 20%, 10% or less of the volume of the native stomach. Surgeons create the neo-stomach by suturing or stapling off a small portion of the stomach near the GEJ and then often severing this connection to the bypassed portion of the stomach. In cases where there is a bypassed portion of the stomach, it is left in the patient. When there is a completely sectioned off portion such as in the sleeve gastrectomy procedure, the excised portion is removed.

With all of these aforementioned bariatric surgery procedures, there are a certain percentage that do not have acceptable outcomes. This can be caused by a number of reasons. Endoscopic examination or other followup diagnostic studies such as barium swallow, CT scan, or MRI can show that the neo-stomach has undesirably expanded, the presence of a fistula leading out of the neo-stomach, and/or the length of the bypass may not be sufficient. In any of these cases, while referred to as an endoscopic bypass sleeve the sleeve could be implanted surgically, laparoscopically, or more preferably endoscopically in some embodiments to accomplish additional weight loss using any of the delivery techniques previously disclosed in the applications previously incorporated by reference, such as, for example. If necessary, the EBS could have modifications or attachments as will be disclosed to address other issues relating to the changes in the post-surgical anatomy. The endoscopic bypass sleeve (also referred to herein as the EBS or bypass sleeve) can be, in some embodiments, a generally tubular sleeve that could be used, in some embodiments, either (1) as an adjunct at the time of the initial bariatric surgical procedure, to create a restriction or a gastric and partial intestinal bypass; or (2) as a re-do procedure after the initial bariatric surgical procedure for patients that have experienced an unacceptable or suboptimal clinical result, to create a restriction, restore a restriction, or to create or lengthen a partial intestinal bypass. Some non-limiting examples of potential uses for a bypass sleeve concurrent with or following a bariatric surgical procedure are listed in the table below.

TABLE 1
Potential Bypass Sleeve Indications with existing Bariatric
Surgical Procedures.

| | Adjunct at the time of the | |
|---|---|---|
| Bariatric Surgical Procedure | procedure | Add on after the procedure |
| Vertical-banded gastroplasty | Place EBS to create a gastric | Place EBS to restore a |
| | and partial intestinal bypass | restriction (e.g., a restrictive |
| | (e.g., a malabsorptive effect) | effect) or to create a gastric |
| | | and partial intestinal bypass |
| Lap-Band | Place EBS to create a gastric | Place EBS to create a gastric |
| | and partial intestinal bypass | and partial intestinal bypass |
| Roux-en-Y gastric bypass | N/A | Place EBS to restore a |
| | | restriction or to lengthen the |
| | | partial intestinal bypass |
| Sleeve Gastrectomy | Place EBS to create a gastric | Place EBS to restore a |
| | and partial intestinal bypass | restriction or to create a |
| | | gastric and partial intestinal |
| | | bypass |
| Biliopancreatic Diversion | Place short EBS to create a | Place EBS to create a |
| | gastric restriction | restriction or lengthen a |
| | | partial intestinal bypass |
| Biliopancreatic Diversion | N/A | Place EBS to restore a |
| with Duodenal Switch | | restriction or lengthen a |
| | | partial intestinal bypass |

If the anastomosis has dilated, the EBS could have a stoma that returns the size of the anastomosis to its desired dimension. The stoma could be located anywhere along the length of the EBS as has been previously described.

If neo-stomach is dilated or expanded, the EBS could have a volume occupying component that will reside in the neo-stomach. This could be, for example, an implant such as an inflatable balloon, expandable mesh, or bezoar type device, such as, e.g., on the outside of the sleeve that can reduce the effective volume of the neo-stomach and induce a sensation of satiety.

In a redo procedure, the size of the proximal anastomosis from the previous bariatric surgery can vary considerably. Because of this, the EBS could come in a variety of sizes to fit the variable anatomy encountered in the procedure. Typical proximal diameters for the EBS could be, in some embodiments, at least about 5 mm to no more than about 50 mm, more preferably at least about 10 mm and no more than about 40 mm. The proximal end would be most preferably sized in some embodiments to fit the neo-stomach or gastroesophageal junction (GEJ) where it will be placed and the sleeve diameter can be selected based on the size of the anastomosis at the time of the procedure. In some embodiments, these two components could be separate pieces that are selected from a range of sizes and fit together during the procedure. Means to connect the proximal end and the sleeve could include snap fit connections, press fit connections, suturing, clipping, stapling, quick setting adhesives or epoxies, thermal bonding, crimping, etc.

Alternatively, the sleeve diameter does not have to be constant throughout the length of the sleeve based on the diameter of the anastomosis diameter. Between the proximal end of the sleeve and the rest of the length of the sleeve could be a neck portion that is sized to fit the anastomosis. If the anastomosis has not dilated much since surgery, it could be less than about 5 cm, 4 cm, 3 cm, 2 cm, 1 cm, or less in diameter. As an example, in one embodiment the proximal end may be 30 mm in diameter, taper down to 10 mm in diameter then expand back to 20 mm in diameter.

The orientation of the anastomosis can vary and should not be considered a limitation for placement of the EBS. The proximal anastomosis, known as the gastro-jejunal or GJ anastomosis, can be side-to-side, end-to-side, or end-to-end. If desired, the EBS need not be a substantially linear tube and could have variations either in the form of curves or elbows along its length to accommodate for these variations. This could occur for example if the proximal end is implanted in the GEJ and then needs to make a sharp turn to go through a side-to-side anastomosis in the neo-stomach connecting to the intestine.

One example of a patient who could benefit from a bypass sleeve as an adjunct to bariatric surgery is a patient has a RYGB and has good results initially including significant excess weight loss. At a certain time, such as 6, 12, or 18 months or more or less the patient begins to regain weight and experience increased hunger. Endoscopic examination shows that the neo-stomach is enlarged and the proximal anastomosis has also expanded. The decision is made to implant an EBS. The EBS, in one embodiment, preferably has a proximal, or top portion that is preferably configured to expand to a greater diameter than the proximal anastomosis, such as at least about 100%, 125%, 150%, 175%, 200%, or more of the diameter of the proximal anastomosis. The proximal portion could be made of a shape memory material such as nitinol, elgiloy, or a polymer, and/or plastic, silicone or other materials, or other materials depending on the desired clinical result in some embodiments. The proximal portion could be a solid structure, a mesh structure, woven, braided or a stent in some embodiments. After the proximal portion which can be made of shape memory material, for example, the rest of the EBS is a bypass sleeve as described in prior applications, but is approximately between about 50-200 cm long in some embodiments, between about 10-20 mm in diameter, such as about 15 mm in diameter, and preferably has a hydrophilic outer coating, such as the HARMONY coating from Surmodics in some embodiments.

### Sleeve Attachments

FIG. 6 illustrates GI anatomy status-post Roux-en-Y bypass. Illustrated is the esophagus 164, gastroesophageal junction (GEJ) 162, neo-stomach 160, neo-stomach staple or suture lines 158, bypassed stomach staple or suture lines 156, bypassed stomach 154 and pylorus 153, duodenum 151, proximal neo-stomach-jejunal anastomosis 152, Roux intestinal limb 150, and distal intestinal anastomosis 149.

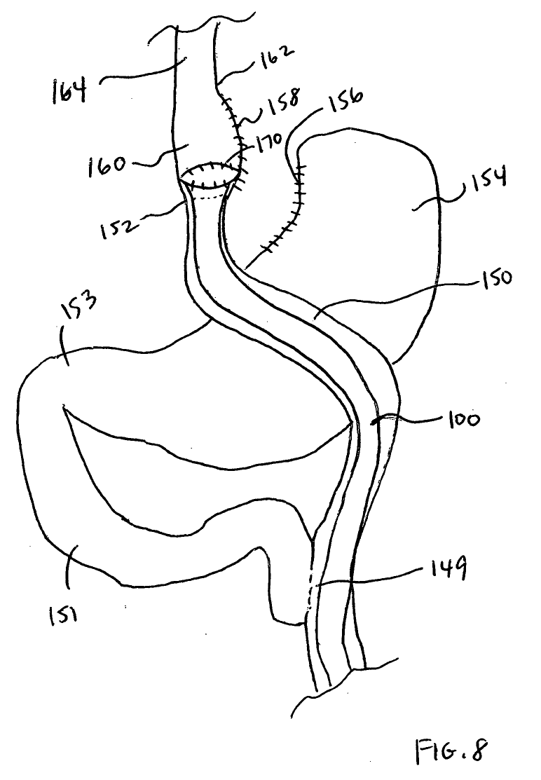

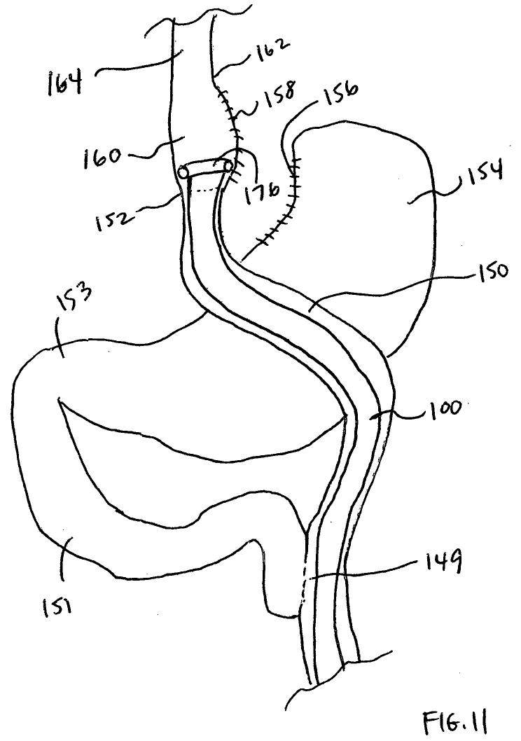

In some embodiments, a gastrointestinal bypass sleeve is proximally attached to a portion of, for example, the esophagus, the gastroesophageal junction, the stomach, or neo-stomach in order to prevent undesired migration of the bypass sleeve. Various non-limiting examples of attachment embodiments will be described in connection with FIGS. 7-17 below. While attachment embodiments as described herein may describe attachment of the sleeve partially or transmurally through at least one luminal wall or as a placation, attachment mechanisms can also be non-penetrating, as shown, for example, in FIGS. 9-12. Furthermore, while attachment mechanisms are described in connection with Roux-en-Y bypass anatomy, it will be understood that the attachment mechanisms, sleeve, and methods can be adapted to anatomy after other bariatric surgical procedures as well, as described elsewhere in the application.

FIG. 7 schematically illustrates an endoscopic bypass sleeve 100 implanted in the proximal stomach (neostomach) 160 attached transmurally through the wall of the neo-stomach 160 to a serosal surface via an attachment cuff 102 and transmural tissue anchors such as T-tags 104 in a patient status-post Roux-en-Y bypass, according to one embodiment of the invention. The attachment cuff can be, for example, as described in connection with FIGS. 1-3 and paragraphs [0111] to [0125] of U.S. Patent Pub. No. 2007/0198074 to Dann et al. (the Dann '074 publication), hereby incorporated by reference in its entirety. Various tissue anchors such as T-tags that can be used, for example, can be found in, e.g., FIGS. 2 and 5A-7B and paragraphs [0126] to [0129] and [0136] to [0157] of the Dann '074 Dann publication, previously incorporated by reference in its entirety. Additional fasteners that can be used are described, for example, in U.S. Provisional Application No. 61/033,385 filed Mar. 3, 2008 and incorporated by reference in its entirety, such as, for example, in FIGS. 1-5 and the accompanying text at paragraphs [0002] to [0022] of the '385 provisional application.

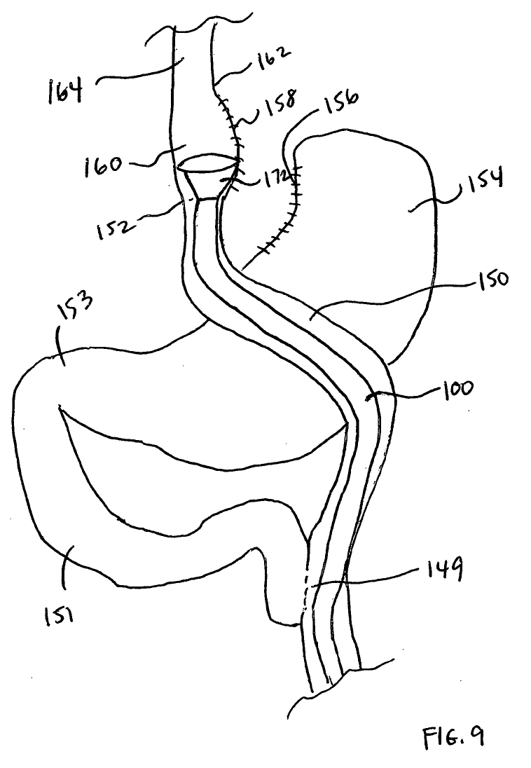

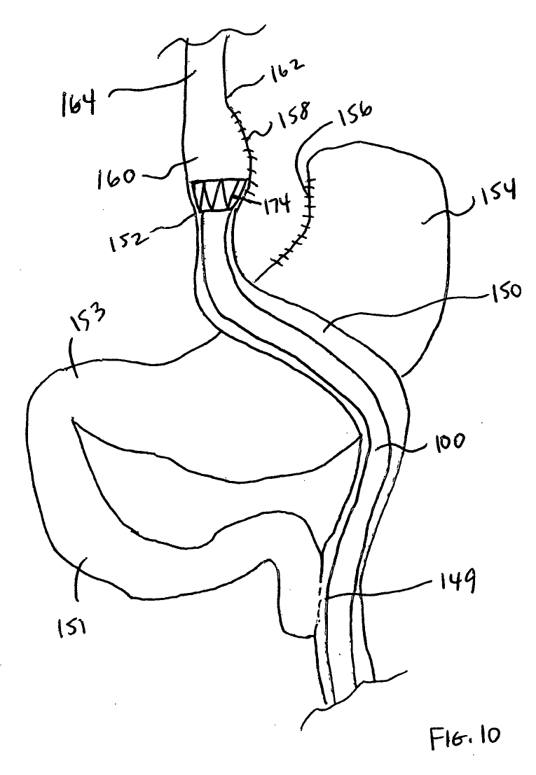

FIG. 8 shows an endoscopic bypass sleeve 100 implanted in the neostomach 160 and attached via staples or sutures 170 through the wall of the neostomach 160 in a patient status-post Roux-en-Y bypass, according to one embodiment of the invention.

FIG. 9 illustrates an endoscopic bypass sleeve 100 implanted in the neostomach 160 attached via an outwardly biased, funnel-shaped structure 172. The structure may be made out of any appropriate biocompatible material, such as, for example, silicone, nitinol, or a shape memory polymer. The proximal end of the funnel-shaped structure 172 preferably has a diameter in its implanted configuration greater than the diameter of the proximal anastomosis 152 as well as the esophagus 164 in order to prevent the funnel-shaped structure 172 connected to sleeve 100 from migrating distally into the Roux limb 150 or proximally back into the esophagus 164.

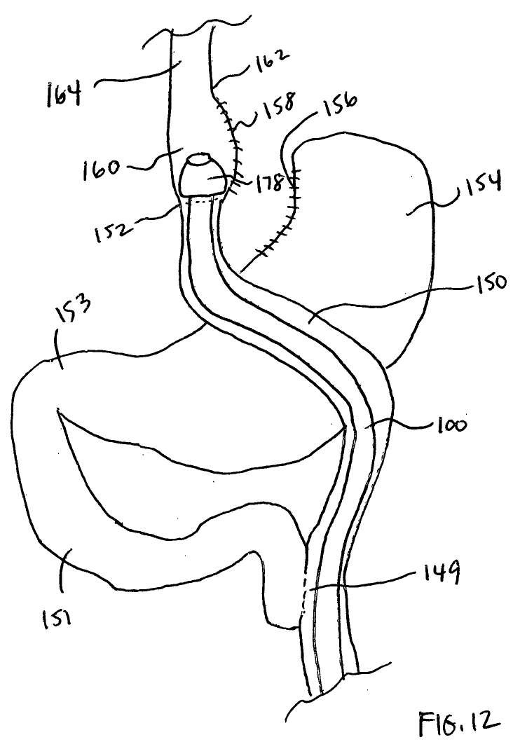

FIG. 10 shows an endoscopic bypass sleeve 100 implanted in the neostomach 160 and secured via an expandable stent 174 in a patient status-post Roux-en-Y bypass, according to one embodiment of the invention. The stent 174 may be made, for example, of nitinol, elgiloy, stainless steel, a shape memory polymer, or other appropriate material.

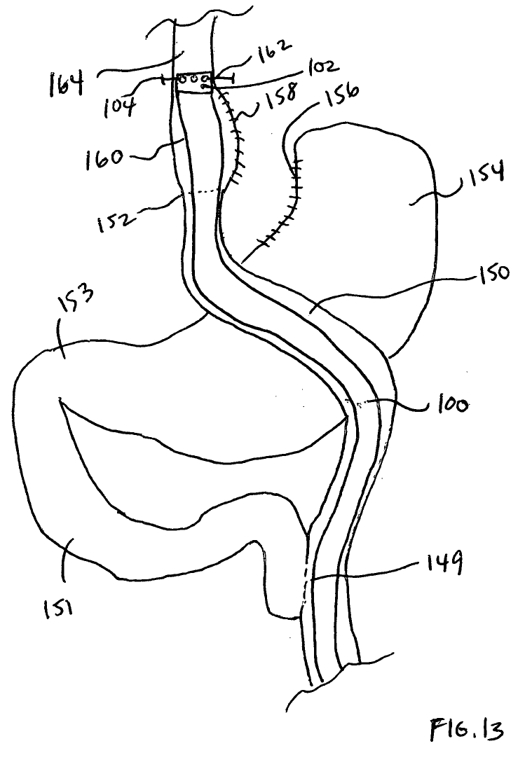

FIG. 11 shows an endoscopic bypass sleeve 100 implanted in the neostomach 160 secured via one or more inflatable or fillable toroidal rings or balloons 176 in a patient status-post Roux-en-Y bypass, according to one embodiment of the invention. The ring or balloon 176 may include a valve or pierceable septum such that it can be filled with a gas, liquid, or solid material.

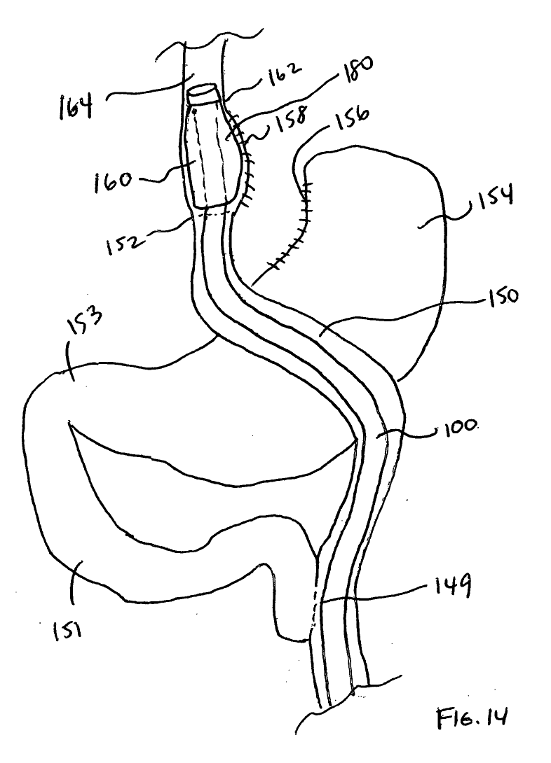

FIG. 12 shows an endoscopic bypass sleeve 100 implanted in the neostomach 160 secured via an expandable dome-shaped element 178 in a patient status-post Roux-en-Y bypass, according to one embodiment of the invention. The dome 178 may be concave, convex, or other shape, and be made of a variety of materials, such as nitinol or other shape memory material, silicone, or other biocompatible material.

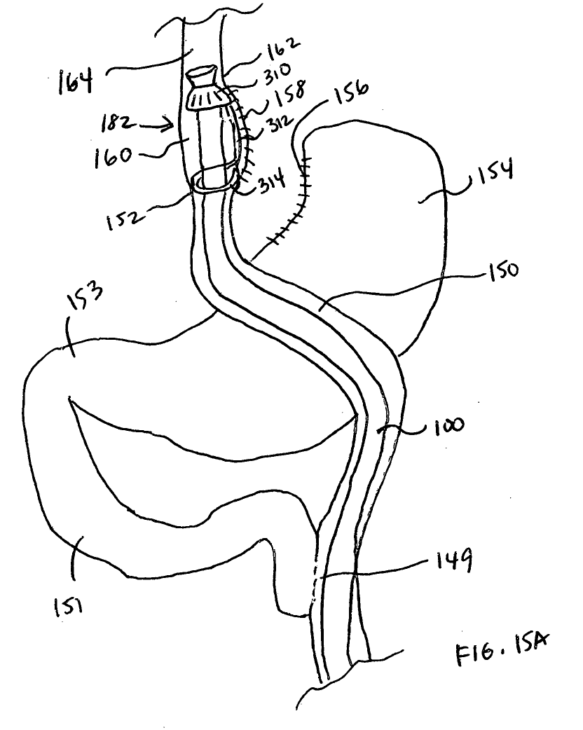

FIG. 13 shows an endoscopic bypass sleeve 100 implanted in the neostomach 160 similar to as shown in FIG. 7, although secured more proximally in the vicinity of, or at the gastroesophageal junction 162, such as with a cuff 102 connected to one or more tissue anchors 104, in a patient status-post Roux-en-Y bypass, according to one embodiment of the invention. Such an attachment site could be advantageous to fully exclude the entire neostomach 160. Furthermore, the relatively thick tissue wall at the gastroesophageal junction 162 may allow for a secure location for transmural attachment.

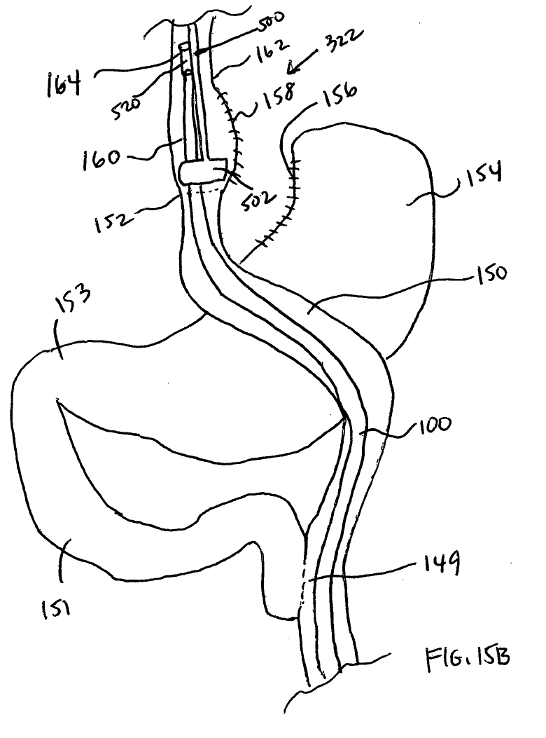

FIG. 14 shows an endoscopic bypass sleeve 100 implanted in the neostomach 160 with an expandable element 180 around, such as at least partially circumscribing, the portion of the sleeve 100 in the neostomach 160 for holding the sleeve 100 in place, in a patient status-post Roux-en-Y bypass, according to one embodiment of the invention.

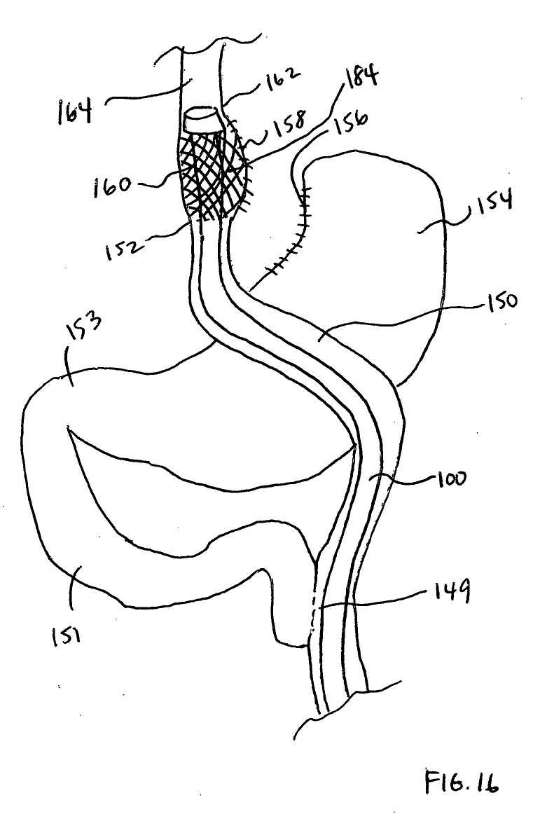

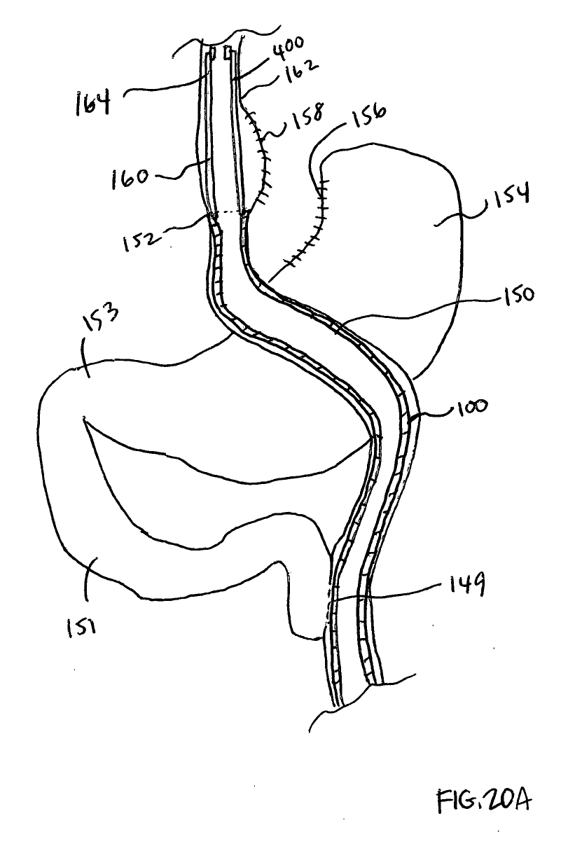

FIG. 15A shows an endoscopic bypass sleeve 100 implanted in the neostomach 160 with an intragastric support system 182 for holding the sleeve in place, in a patient status-post Roux-en-Y bypass, according to one embodiment of the invention. The intragastric support system 182 includes a proximal dome-shaped element 310 that can be configured to have a diameter greater than the diameter of the distal esophagus 164, to prevent proximal migration out of the neo-stomach 160 and into the esophagus 164. The system also includes an intragastric support element 312 that may include an arcuate element 316 and a distal loop 314 with an outside diameter larger than the diameter of the distal neo-stomach to prevent distal migration of the device out of the neo-stomach and into the Roux loop 150. The system may also include a proximal orientation element 500 (not shown) that may be an elongate member extending into the esophagus to stabilize the device. Other intragastric support systems that do not necessarily require attachment through a wall of the GI tract to maintain the position of a sleeve that can be used or modified or use with those described herein are disclosed, for example, at FIGS. 1A-6D and 11-13C and the accompanying text at paragraphs [0029] to [0057] of U.S. Provisional Application No. 61/023,809, hereby incorporated by reference in their entirety, as well as in PCT Application No. PCT/US2008/066214 filed Jun. 6, 2008 and incorporated by reference herein in its entirety, such as, for example, at FIGS. 1A-17A and the accompanying text at paragraphs to [0127]. Delivery methods that can be used or adapted for use with intragastric support systems can be found, for example, at FIGS. 18A-21C of PCT/US2008/066214 and the accompanying text at paragraphs [0128] to [0141].

FIG. 15B illustrates a bypass sleeve 100 attached proximally to a collecting ring 520 of an intragastric support system 322, according to another embodiment of the invention. The system 322 can include at least two elements: (1) a proximal orientation element 500 and (2) a distal support element 502, as shown. The proximal orientation element 500 can be configured to reside at least partially within the esophageal lumen and the distal support element 502 can be configured to reside in the stomach or neo-stomach 160. The proximal orientation element 500 may be integrally formed with the distal support element 502, or separately formed and coupled, such as via a joint such as, for example, a hinge or ball-and-socket joint. The distal support element 502 could be unitary, or include several different subcomponents in other embodiments. The system can have a first configuration in which the long axis of the proximal orientation 500 element is coaxial or substantially coaxial with the long axis of the distal support element 502, and a second configuration in which the long axis of the proximal orientation element 500 is not substantially coaxial with the long axis of the distal support element 502. The second configuration can thus advantageously retain the gastrointestinal support system in place and prevent unwanted proximal migration of the gastric support element 502 into the esophagus or distal migration into the intestine, as the distal support element 502 has at least one diameter that is larger than a diameter of the distal esophagus and pylorus, respectively. In some embodiments, in the second implanted configuration of the gastrointestinal support system, the long axis of the gastric support element and the long axis of the esophageal strut element intersect and form an angle of between about 30-90 or 45-75 degrees, or are perpendicular or substantially perpendicular in other embodiments. The system can transform from the first configuration to the second configuration via a variety of mechanisms, such as, for example, actuation of a hinge or ball-and-socket joint between the proximal orientation element 500 and distal support element 502, deformation of the distal support element 502 via bending or shape memory material, expansion via, e.g., a balloon or expandable polymer. The system can, in some embodiments, transform from the second non-coaxial configuration back into the first coaxial configuration to promote removal of the system from the body lumen.

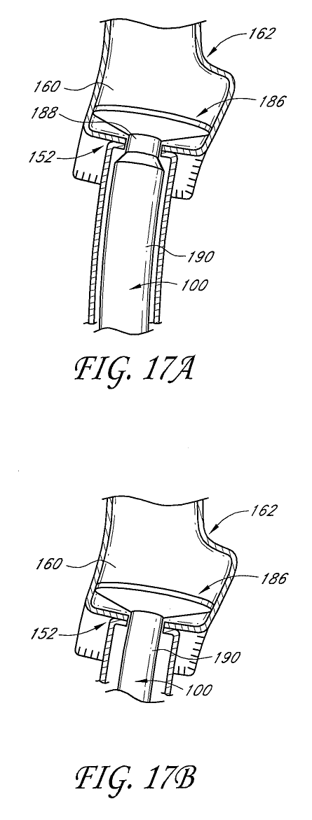

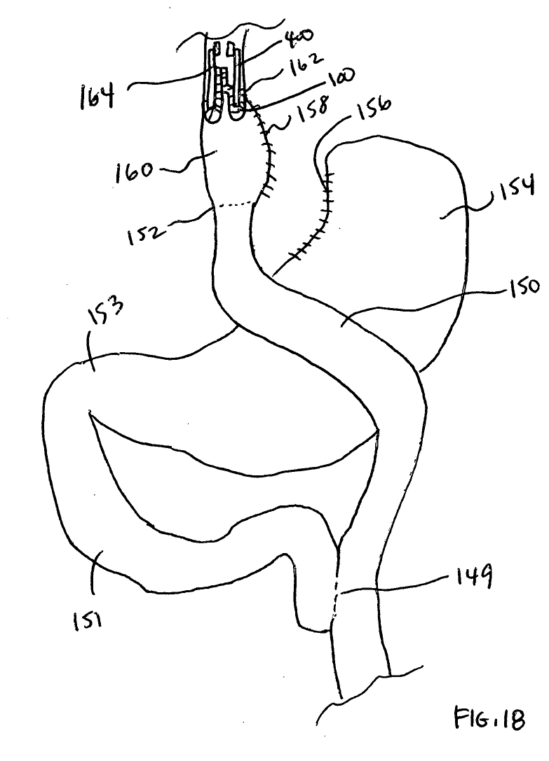

FIG. 16 shows an endoscopic bypass sleeve 100 implanted in the neostomach 160 with an expandable woven shape memory material portion 184 within the neostomach for holding the sleeve in place in a manner somewhat similar to the attachment devices described and illustrated in FIGS. 9-12 and 14 above, in a patient status-post Roux-en-Y bypass, according to one embodiment of the invention.

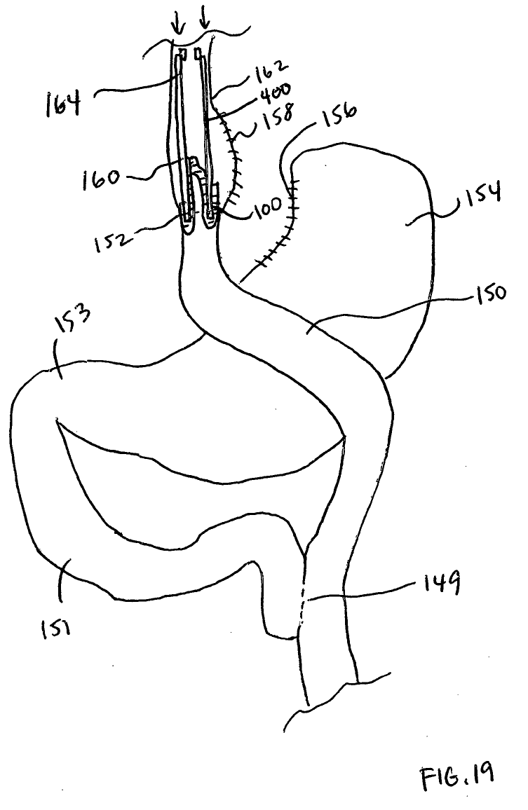

FIG. 17A illustrates a close-up cross-sectional view of a bypass sleeve 100 with an outwardly biased proximal portion 186 and a neck portion 188 with a diameter smaller than that of a diameter of the distal remainder of the sleeve 190, and configured to operably engage the proximal Roux-en-Y anastomosis 152, according to one embodiment of the invention. A diameter of the neck portion can be, in some embodiments, no more than about 15, 10, 8, 6, 5, 4, 3, 2, 1 cm, or less.

FIG. 17B illustrates another embodiment similar to that of FIG. 17A except the diameter of the neck portion 188 is the same or substantially the same as that of the distal remained of the sleeve 190.

### Delivery Methods

A method for implanting a bypass sleeve 100 within a patient is disclosed and illustrated in FIGS. 18-20B, according to some embodiments of the invention. The top portion of the bypass sleeve 100 is placed around the distal end of a delivery catheter 400 and the 100 sleeve is inverted up through the center, as shown in FIG. 18. An overtube (not shown) can be deployed prior to insertion of the delivery catheter to prevent trauma to anatomical structures.

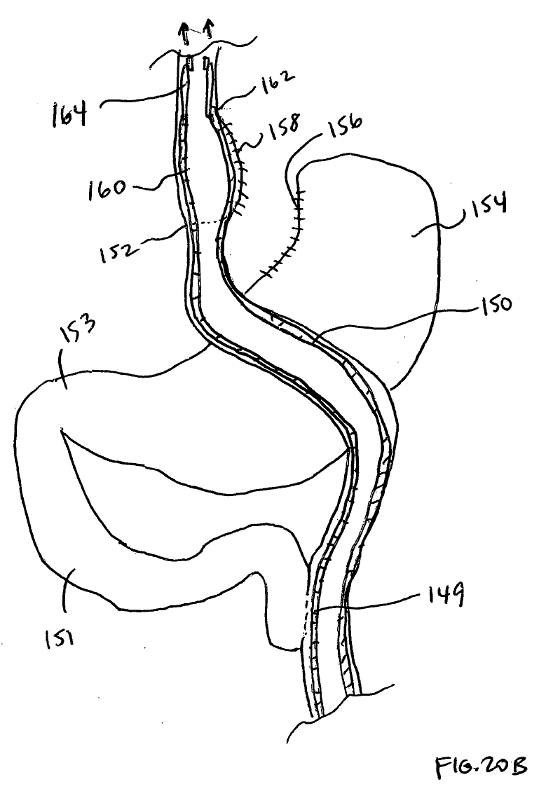

The delivery catheter 400 is advanced perorally into the neo-stomach 160 and cannulates the GJ anastomosis 152, as illustrated in FIG. 19. Next, the bypass sleeve 100 is deployed toposcopically down the Roux limb 150 of the Roux-en-Y gastric bypass, as illustrated in FIG. 20A. Additional details regarding toposcopic delivery of a gastrointestinal sleeve 100 may be as described, for example, in U.S. patent application Ser. No. 11/861,156 filed Sep. 25, 2007, and hereby incorporated by reference in its entirety. More specifically, for example, FIGS. 1A-2E of the 11/861,156 application and the accompanying text at paragraphs [0054] to [0064] disclose various embodiments of toposcopic sleeves; FIG. 15H and the accompanying text at paragraph [0143] disclose an embodiment of a filling catheter and sleeve kit; and FIGS. 3A-16B and the accompanying text at paragraphs [0065] to and [0144] to [0150] disclose various toposcopic delivery systems and components including collapsible and steerable filling catheters, guidewires, techniques for occluding the distal end of the sleeve, and loop snares, all of which can be used or modified for use with the systems and methods described herein.

The delivery catheter 400 is then retracted into the neo-stomach 160 as illustrated in FIG. 20B and can be attached, for example, as previously illustrated in FIGS. 7-17B above. In some embodiments, a shape memory proximal attachment portion, e.g., as described in connection with FIG. 15, is inserted at the level of the GEJ, neostomach, or proximal anastomosis and released. Then, the shape memory top may have enough bias to hold in place above the proximal anastomosis or it could be tagged in place via endoscopic T-tags, suturing, or anchor placement. These attachments could be transmural or non-transmural (e.g., non-penetrating), such as, for example, any of the attachment mechanisms described in connection with FIGS. 7-17B above. If, at a later date, the physician decides the bypass sleeve 100 needs to be removed, this can be achieved perorally as well by, for example, cutting sutures or removing staples attaching the top of the bypass sleeve 100 and grasping the proximal end with an endoscopic grasper and then removing perorally.

Other various ways can be used to attach the proximal end of the bypass sleeve to the desired location. These include: T-tags, sutures, adhesives, such as PMMA, cyanoacrylate, or fibrin glue; stents, such as cylindrical or conical shaped stents or z-stents; barbs, shape memory funnels, shape memory gasket or baffle elements, that may be dome-shaped in some embodiments, balloons, expandable mesh structures such as woven or braided nitinol, elgiloy or plastics that can be optionally coated with a material such as silicone, polyurethane, ePTFE, or PTFE. The proximal end can also include an intragastric support sized to fit the neostomach. Various possible attachment options are described in FIGS. 7-17B and described above. While the Figures are shown with respect to a Roux-en-Y bypass, one of ordinary skill will appreciate that the length and configuration of the sleeve can be modified for other bariatric procedures such as those described herein, and need not necessarily extend from the stomach or neostomach to the jejunum; shorter and longer sleeves are also within the scope of the invention.

In many cases the neo-stomach has much less motility than a normal stomach, and so attachment to this area may be able to use devices that would not work effectively in unmodified (native) anatomy.

In some embodiments, the bypass sleeve may be implanted in the patient for at least about 1 week, 2 weeks, 3 weeks, 4 weeks, 3 months, 6 months, 1 year, 2 years, 3 years, 5 years, or more depending on the desired clinical result. In some embodiments, the bypass sleeve may be switched out after any time period for a longer or shorter bypass sleeve if clinically indicated.

So far, what has been described primarily has been attaching the bypass sleeve 100 in the neo-stomach 160 above the proximal anastomosis 152. The bypass sleeve 100 could also be attached at the top of or above the neo-stomach 160 at or near the GEJ 162 using any of the described attachment methods. This may be determined based on the size of the neo-stomach 160 at the time of the redo procedure. If the neo-stomach 160 has dilated since the initial bariatric procedure, placement of the proximal end of the bypass sleeve higher (more proximal) in the neo-stomach 160 or at the GEJ 162 would reduce the volume by partitioning the neo-stomach 160 to serve to create a smaller volume.

In addition, in some embodiments, the bypass sleeve 100 could be deployed below the proximal anastomosis 152, for example, in the proximal part of the ROUX Limb 150.

Note that in a sleeve gastrectomy, since there is no anastomosis, the preferred location in some embodiments, for attachment would be at or near the GEJ 162, but could be placed further down (distally) in the neo-stomach 160 in other embodiments.

The length of the bypass sleeve 100 could be varied either for a desired length or to have a desired placement relative to the anatomy. For example, it may be beneficial to have the length of the sleeve 100 less than the distance to the distal anastomosis, known as the jejunaljejunal or JJ anastomosis, greater than that length or equal to that length, depending on if it is desired to adjust the location where the food contents in the sleeve mix with the bilio-pancreatic secretions coming out of the duodenum and bypassed portion of the stomach.

### Dimensions

At the time of surgery, the size of the neo-stomach varies depending on the procedure:

RYGB—typically the volume is 20-30 ml and the size of the GJ (proximal) anastomosis is 10 mm

SG—the diameter of the sleeve is typically created by placing a tube into the stomach and using that as a mandrel to create the sleeve and target the staple line. The typical diameter of the tube used is 10-15 mm. The resulting neo-stomach is in the range of 30% or less of the normal stomach.

Some estimates indicate that 20-30% of AGB patients do not achieve satisfactory weight loss. Many of these patients will go on to have a more invasive procedure such as the Roux-en-Y gastric bypass. The sleeve device could be used in conjunction with one or more gastric bands. The band would be left in place and forms a stoma at the GEJ. The bypass sleeve would be deployed in the stomach and intestine as described previously. The proximal part of the sleeve would be attached just above the stomach, in the ways described previously. In the way, the adjustable feature of the gastric band could still be used, altering the stoma size as needed.

Sleeve material and embodiments, for example, can be as described in previous disclosures, such as disclosed in the Kagan '892 publication, for example, at FIGS. 11-31 and the accompanying disclosure at, e.g., paragraphs [0241] to [0312] of the publication, or, for example, at paragraphs [0174] to [0185] of the Dann '074 publication, both of which are incorporated by reference in their entirety. The sleeve can have a stiffening element to keep the sleeve in place after delivery. The stiffening element has been described in the Kagan '148 application and could be a wire built into the wall of the sleeve or there could be a lumen in the wall of the sleeve that can be filled with a stiffening material as previously described.

Thus far described herein is placing the bypass sleeve in a mode of revisional or failed primary bariatric surgery. The device as described herein and in related applications could, in some embodiments, be placed either at the time of bariatric surgery in any of the procedures described or at a later time if results with the primary procedure are not satisfactory.

If the bypass sleeve is deployed at the time of surgery when an intestinal bypass is constructed, it could be done either perorally or operatively during the procedure. In a RYGB or VBG-GB, the bypass sleeve could be deployed into the ROUX limb before it is connected to the neo-stomach.

If placed during the procedure the bypass sleeve could be used to create a longer section of bypassed intestine. For instance, if the physician desired in a RYGB or VBG-GB to bypass 150 cm of intestine, they could create the physical bypass via the surgery at 75 cm and then place a 75 cm EBS at the proximal anastomosis as shown in FIG. 6. This would have the similar effect to a 150 cm bypass, however it would not be permanent. If after a few months weight loss was satisfactory, however the patient developed a nutritional deficit or other complication the physician could perform a per-oral procedure to remove the sleeve. This would return the patient to a 75 cm bypass and may relieve the complications. This could be used to create a very short bypass where the distal anastomosis connects to the lower part of the duodenum and the bypass sleeve goes on for 40-100 cm. Because many nutrients such as vitamins and minerals are absorbed in the duodenum, the ability to remove the EBS and expose the lower part of the duodenum to nutrients again may be very beneficial. Various sleeve lengths can be used depending on the desired clinical result. In some embodiments, the sleeve is no more than about 150 cm, 140 cm, 130 cm, 120 cm, 110 cm, 100 cm, 90 cm, 80 cm, 70 cm, 60 cm, 50 cm in length, or less.

While the present application describes various sleeve lengths and features in the context of certain bariatric surgical procedures, the sleeve can be of any desired length depending on the desired clinical result. For example, in some embodiments, the sleeve when positioned can extend proximally from the esophagus or GEJ distally to the duodenum, jejunum, ileum, or colon, or any between any other two locations within the GI tract. The sleeve may, in some embodiments, have an axial length of at least about 20 cm, 30 cm, 40 cm, 50 cm, 75 cm, 100 cm, 125 cm, 150 cm, 175 cm, 200 cm, 225 cm, 250 cm, 275 cm, 300 cm, 350 cm, 400 cm, 450 cm, 500 cm, 550 cm, 600 cm, 700 cm, 800 cm, 900 cm, or more. In other embodiments, the sleeve may have an axial length of no more than about 900 cm, 800 cm, 700 cm, 600 cm, 550 cm, 500 cm, 450 cm, 400 cm, 350 cm, 300 cm, 275 cm, 250 cm, 225 cm, 200 cm, 175 cm, 150 cm, 125 cm, 100 cm, 75 cm, 50 cm, 40 cm, 30 cm, 20 cm, or less.

The bypass sleeve may be delivered toposcopically as described elsewhere in the application. In some embodiments, the sleeve can be delivered toposcopically as described in U.S. patent application Ser. No. 11/861,156 entitled TOPOSCOPIC ACCESS AND DELIVERY DEVICES, filed Sep. 25, 2007, and hereby incorporated by reference in its entirety. The proximal end of the sleeve may be secured to the stomach or esophagus, such as the GEJ as described, using both transmural and non-transmural attachment methods as described, for example, in connection with FIGS. 7-17B above.

Common Nutritional Deficiencies are:

Hyperparathyroidism, due to inadequate absorption of calcium, may occur in a large number, such as over 30% of GBP patients. Calcium is primarily absorbed in the duodenum, which is bypassed by the surgery. Most patients can achieve adequate calcium absorption by supplementation with Vitamin D and Calcium Citrate (carbonate may not be absorbed—it requires an acidic stomach, which is bypassed).

Iron frequently is seriously deficient, particularly in menstruating females, and should be supplemented. Again, iron is normally absorbed in the duodenum. Ferrous sulfate can cause considerable GI distress in typically prescribed doses; alternatives include ferrous fumarate, ferrous gluconate, or a chelated form of iron. Occasionally, a female patient develops severe anemia, even with supplements, and must be treated with parenteral iron.

Vitamin B-12 requires intrinsic factor from the gastric mucosa to be absorbed. In patients with a small gastric pouch, it may not be absorbed, even if supplemented orally, and deficiencies can result in pernicious anemia and neuropathies. Sub-lingual B-12 appears to be adequately absorbed.

Thiamine deficiency (also known as beriberi) will, rarely, occur as the result of its absorption site in the jejunum being bypassed. This deficiency can also result from inadequate nutritional supplements being taken post operatively.

Protein malnutrition is a significant risk in patients post-bariatric surgery. Some patients suffer troublesome vomiting after surgery, until their GI tract adjusts to the changes, and cannot eat adequate amounts even with several meals a day. Many patients require protein supplementation during the early phases of rapid weight loss, or even parenteral nutrition, to prevent excessive loss of muscle mass.

Note that if so desired the bypass sleeve could be used to replace the intestinal bypass or create one, while the neo-stomach is still created with a surgical technique. An example would be in the SG. In this procedure a neo-stomach approximately 30% of the volume of the native stomach is created, but here there is no intestinal bypass. By placing the bypass sleeve either in the neo-stomach or at the GEJ the procedure would have the added benefit of an intestinal bypass without the permanent alteration to the anatomy. An intestinal bypass is illustrated in FIG. 16 of the Kagan '148 application and FIG. 1A of the Kagan '634 application, incorporated by reference as noted above. One particular example of a bypass sleeve to replace an intestinal bypass, where the neo-stomach is still created using a surgical technique is described below.

Bypass Sleeve Placement as Alternative to Biliopancreatic Diversion with or without Duodenal Switch following Sleeve Gastrectomy

The sleeve gastrectomy, Magenstrasse and Mill procedure, or other procedure such as those described and illustrated above may be followed by a second stage procedure, such as a biliopancreatic diversion (BPD) with or without duodenal switch (DS or BPD-DS), typically 6-12 months following the sleeve gastrectomy procedure. The delay prior to the second stage procedure can allow the physician a period of time to evaluate the patient's clinical course following the first bariatric surgical procedure and determine if a second intervention is indicated to promote additional weight loss.

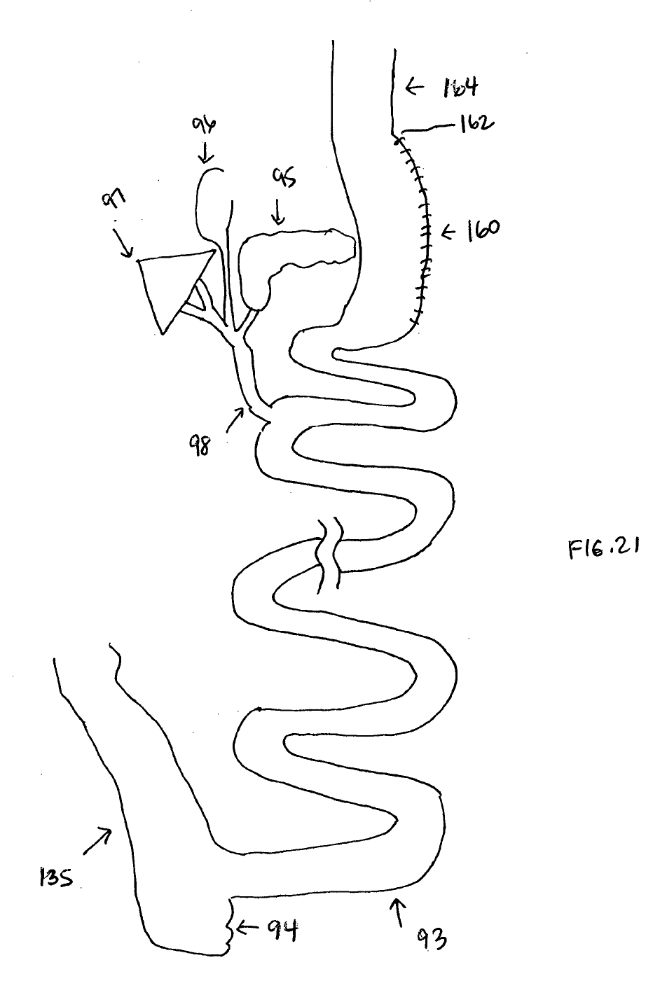

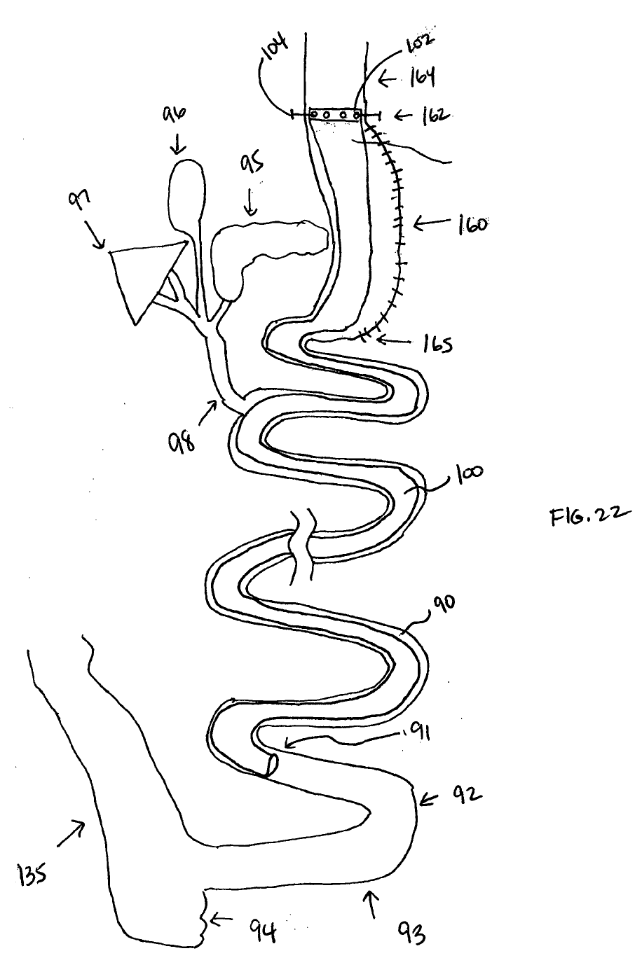

Anatomy of the gastrointestinal tract following sleeve gastrectomy is shown in FIG. 4 above. As shown in FIG. 4, the sleeve gastrectomy is a procedure in which the stomach is reduced to approximately one-third of its native size by removal of a portion of the stomach following the greater curve. The remaining free edges of the stomach are attached together, e.g., using staples, to form a sleeve or tube, while anatomy after a Magenstrasse and Mill procedure is shown in FIG. 5B. A further schematic (showing the intestinal tract as well) after sleeve gastrectomy alone is shown in FIG. 21, illustrating the esophagus 164, GEJ 162, neo-stomach 160, liver 97, gallbladder 96, pancreas 95, common bile duct 98, ileum 93, cecum 94, and large intestine 135.

A biliopancreatic diversion procedure without duodenal switch (BPD, also known as the Scopinaro procedure) is illustrated in FIG. 5C. The lower stomach is removed leaving a pouch of between 250 and 400 cc. The duodenum is closed over. The small bowel is measured backwards from the point where it joins the large bowel or colon a distance of approximately 250 cms and the small bowel is cut across at this point. The lower divided end is joined to the stomach pouch. The bottom end of the upper section is joined to the lower loop so as to make a new opening 50 cm from the point where it joins the colon. In a BPD without DS procedure, the distal stomach and pylorus is not spared, potentially resulting in an increased risk of complications related to absence of the pyloric valve, such as gastric dumping syndrome.

In contrast to the Scopinaro BPD procedure, the DS procedure keeps the pyloric valve intact. As shown in FIG. 5A above, in a DS procedure, a lengthy portion of the small intestine is re-routed to cause a malabsorptive effect. Two separate pathways 136, 138 and one common pathway 139 are created by the procedure. The shorter of the two pathways, the digestive loop 136, which can be, for example, between about 100-250 cm, takes food from the stomach to the distal small intestine, the ileum. The much longer pathway, the bilio-pancreatic loop 138, carries bile from the liver to the common path 139. The common path 139, or common channel, is a stretch of small intestine (e.g., part of the ileum) usually about 75-150 centimeters long in which the contents of the digestive path 136 mix with the bile from the bilio-pancreatic loop 138 before emptying into the large intestine 135. The objective of this arrangement is to reduce the amount of time the body has to capture calories from food in the small intestine and to selectively limit the absorption of fat.

The malabsorptive element of the DS requires that those who undergo the procedure take vitamin and mineral supplements above and beyond that of the normal population, as do patients having the Roux-en-Y surgery. Commonly prescribed supplements include a daily prenatal and vitamin and extra calcium citrate. However, the duodenal switch procedure bypasses a greater proportion of the intestine than the RNY procedure described above and thus allows for considerably less absorption, and thus potential weight loss.

Because gallstones are a common complication of rapid weight loss following any type of weight loss surgery, some surgeons may remove the gallbladder as a preventative measure during the DS or the RNY. Others will retain the gallbladder, and prefer to prescribe medication (e.g., ursodiol) to reduce the risk of post-operative gallstones. In addition to nutritional deficiencies caused by malabsoprtion, potential complications of a BPD with or without DS procedure include anastomostic failure causing leaks, infection, perforation, venous thrombo-phlebitis, ulcers, adhesions, and stomal stenosis.

These complications may be minimized or avoided by an implantable sleeve such as described above that can functionally recreate a BPD-DS. A schematic of a sleeve functionally recreating a DS, shown delivered within the GI tract, according to one embodiment of the invention, is shown in FIG. 22. While a DS procedure may technically be able to be reversed, it cannot do so as easily as a bypass sleeve 100 (which does not affect normal intestinal anatomy) acting as the functional equivalent of a DS. The bypass sleeve 100 within the intestine carrying food can functionally serve as the equivalent of the “digestive loop” of the DS, while the outside of the sleeve 90 within the jejunum and proximal ileum carrying biliary secretions can serve as the equivalent of the “bilio-pancreatic loop.” The portion of the ileum distal to the distal end of the sleeve and proximal to the cecal valve thus acts as the common channel 93 allowing food to mix with digestive juices within the relatively short common channel 93, allowing the digestive juices relatively little time to digest the food and absorb calories from it into the body. As with the DS and in contrast to the Scopinaro BPD without DS procedure, the pylorus 165 is advantageously spared when the bypass sleeve 100 is implanted, which can reduce the incidence of dumping syndrome.

Because the DS switch procedure bypasses a greater proportion of the intestine than the RNY and thus allows for considerably less absorption as noted above, it is generally desirable to use a sleeve having a greater length than a sleeve configured for placement after RNY. A RNY sleeve, in some embodiments, typically has its distal end positioned within the jejunum, at least about 350 cm, 400 cm, 450 cm, 500 cm, 600 cm, 700 cm, or more in a proximal direction with respect to the cecal valve. In contrast, for such a sleeve being implanted to replicate a DS, in some embodiments, the proximal end of the sleeve is secured within the esophagus or stomach, such as at the GEJ, and the distal end of the sleeve is positioned within the ileum, approximately no more than about 200 cm, 175 cm, 150 cm, 125 cm, 100 cm, 75 cm, 50 cm, 40 cm, 30 cm, or less in a proximal direction with respect to the cecal valve. Depending on the desired clinical result, the sleeve could later be replaced by another sleeve of either shorter or longer length to adjust the length of the common channel, and thus the amount of food absorption.

Bypass Sleeve Placement Concurrent with Sleeve Gastrectomy

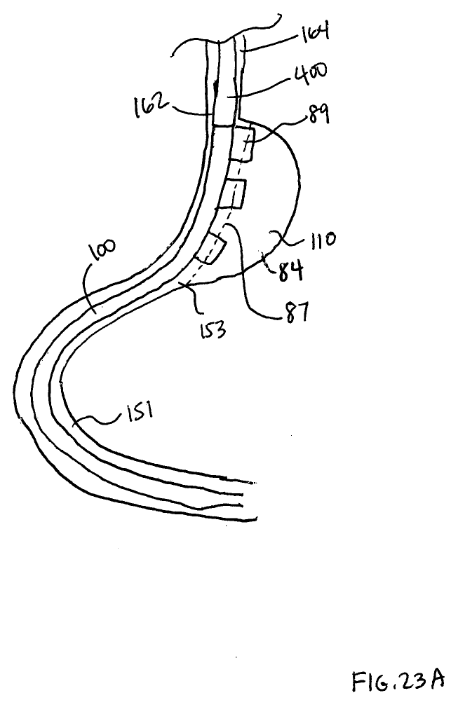

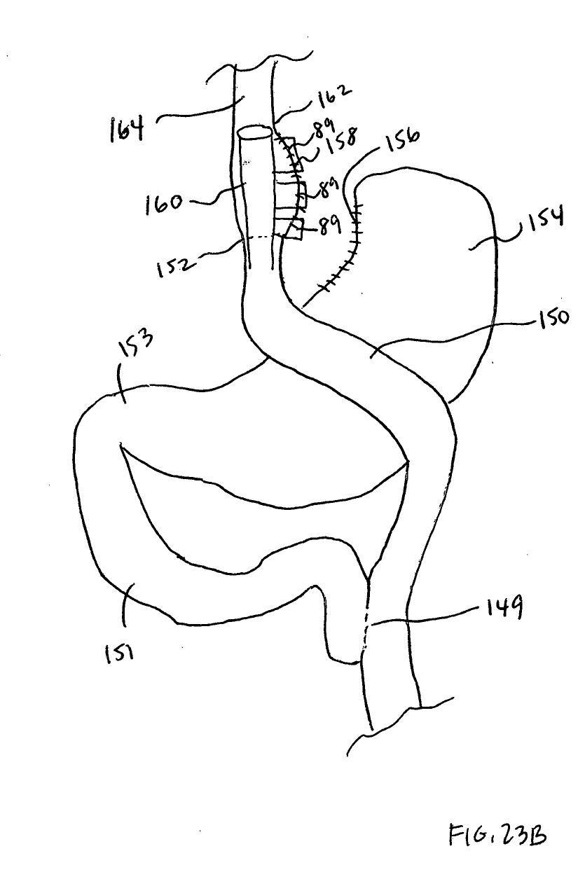

In some embodiments, a sleeve as previously described could be designed specifically for use for placement concurrently with a bariatric surgical procedure, such as sleeve gastrectomy. This may involve the sleeve having expandable or self expanding components such as stents, flanges, rings or other structures made out of, for example, superelastic material. In one embodiment, as shown in FIG. 23A, the sleeve 100 illustrated is configured for being placed in a patient concurrently with a sleeve gastrectomy procedure. The sleeve 100 shown has one or more radial fins 89, such as at least about 2, 3, 4, 5, 6, 7, 8, or more fins, that protrude radially, such as perpendicularly to the long axis of the sleeve within the stomach, from the outer diameter of the sleeve along a portion of its proximal length, as illustrated. The fins 89 can be any appropriate size to fit within the neo-stomach 160, such as 1-10 cm or 1-5 cm in length in some embodiments, and in one embodiment a single elongate fin 89 can span the entire or nearly the entire length of the neo-stomach 160. The fins 89 can be positioned in the stomach 110 prior to stapling of the stomach to reduce the effective stomach volume during sleeve gastrectomy, and thus be attached at the staple line 87 (illustrated in phantom) after stapling such that the sleeve 100 is securely attached in the neo-stomach after the procedure as illustrated in FIG. 23B.

The fins 89 could be made out of a variety of materials. They could be made out of, for example, PTFE, ePTFE, polyurethane, silicone or a variety of materials that are currently used for buttressing staple lines. These buttress materials are commonly used to help prevent leaks along a staple line in surgical procedures, especially in bariatric surgery and can be made by synthetic materials or using biologically-based materials such as pericardial tissue, small intestinal submucosa (SIS), or collagen. This way, the fin 89 may serve a dual purpose of holding the sleeve 100 in place and also helping ensure against staple line leaks.

In some embodiments, the fins 89 would be collapsible for sleeve delivery then expand after delivery of the sleeve or could be released by the operator by actuating a control element. The control element could be as simple as a suture that is wrapped around the fin holding it flat against the previously described delivery catheter. The control suture would hold the fins flat with knots that release when pulled with the control suture.

In some embodiments, the sleeve 100 could have additional structural components to help its function, including a proximal opening with some reinforcement to help keep it in proper alignment to catch incoming food or liquids. This could be any one of the many designs of a cuff with some hoop strength that have been disclosed in previous applications incorporated by reference herein for use at the GEJ with a sleeve. This could include an elastic ring or band at the proximal sleeve opening made from superelastic material or silicone.

A method of deploying a sleeve 100 including one or more fins 89 during a sleeve gastrectomy procedure is described. The sleeve 100 is placed over the delivery catheter so the most distal edge of the most distal fin 89 is proximal of the distal end of the delivery catheter. The sleeve 100 is then inverted into the delivery catheter as previously described. An overtube can then be deployed. Next, the delivery catheter with the sleeve 100 is placed into the pylorus and the sleeve is deployed into the intestine, such as toposcopically as previously described. The fin 89 is then released, expanding radially into the stomach 110 and oriented towards the greater curve 84 of the stomach 110. In some embodiments there are indicia inside the delivery catheter so by placing an endoscope into the delivery catheter the proper position and/or orientation of the fins 89 within the stomach 110 can be confirmed. Alternatively there could be indicia made on the proximal end of the delivery catheter or overtube so by observing the indicia outside of the patient's mouth the operator can confirm the positioning and alignment of the fins 89. The delivery catheter is left at the pylorus for the sleeve gastrectomy. The delivery catheter would serve as the bougie, which is a tube often used in sleeve gastrectomy procedures to size the diameter of the sleeve 100 the surgical procedure will create.

The surgeon then resects the stomach with staples as is currently done in sleeve gastrectomy. The staple line captures the fins of the sleeve holding it the sleeve in place. The sleeve is then released from the delivery catheter and the delivery catheter is removed leaving the sleeve in place.

In some embodiments, the sleeve and fin can be disconnected from the staple line at a later time with an endoscope so that the sleeve could be removed at a later point in time if the desired weight loss is achieved or there is resolution of comorbidities that are intended to be treated, such as diabetes.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially. While any above-listed applications may have been incorporated by reference for particular subject matter as described earlier in this application, Applicants intend the entire disclosures of the above-identified applications to be incorporated by reference into the present application, in that any and all of the disclosures in these incorporated by reference applications may be combined and incorporated with the embodiments described in the present application.

We claim:

1. A method of treating a patient, comprising the steps of:
identifying a patient who has undergone a bariatric surgical procedure; and
positioning a gastrointestinal sleeve device with a proximal end, distal end, and elongate body in a patient such that the proximal end of the sleeve device is positioned in the stomach and the distal end of the sleeve device is positioned in the intestine.

2. The method of claim 1, wherein the stomach is the neo-stomach.

3. The method of claim 1, wherein the bariatric surgical procedure is a Roux-en-Y gastric bypass.

4. The method of claim 1, wherein the bariatric surgical procedure is a sleeve gastrectomy.

5. The method of claim 1, wherein the bariatric surgical procedure is selected from the group consisting of: sleeve gastrectomy with duodenal switch, biliopancreatic diversion, biliopancreatic diversion with duodenal switch, vertical banded gastroplasty, vertical banded gastroplasty with gastric bypass, Lap-Band procedure, and the Magenstrasse and Mill procedure.

6. The method of claim 1, further comprising the step of securing the proximal end of the gastrointestinal sleeve with respect to the neo-stomach.

7. The method of claim 1, wherein the gastrointestinal sleeve device is operably attached to a proximal attachment element at the proximal end of the sleeve device.

8. The method of claim 2, wherein securing the proximal end of the gastrointestinal sleeve comprises penetratingly attaching the sleeve device through a wall of the neo-stomach.

9. The method of claim 2, wherein securing the proximal end of the gastrointestinal sleeve comprises non-penetratingly attaching the sleeve device within the neo-stomach.

10. The method of claim 7, wherein the proximal attachment element comprises an attachment cuff.

11. The method of claim 7, wherein the proximal attachment element comprises an expandable dome.

12. The method of claim 7, wherein the proximal attachment element comprises an outwardly-biased funnel.

13. The method of claim 7, wherein the proximal attachment element comprises a stent.

14. The method of claim 7, wherein the proximal attachment element comprises an expandable structure.

15. The method of claim 14, wherein the expandable structure comprises a balloon.

16. The method of claim 14, wherein the expandable structure comprises a woven shape memory material.

17. The method of claim 1, wherein positioning the distal end of the sleeve device within the intestine comprises toposcopically everting the sleeve within the intestine.

18. The method of claim 1, further comprising the step of leaving the gastrointestinal sleeve device within the patient for at least about 2 weeks.

19. The method of claim 1, wherein positioning the distal end of the sleeve device within the intestine comprises positioning the distal end of the sleeve device within the ileum.

20. The method of claim 1, wherein positioning the distal end of the sleeve device within the intestine comprises positioning the distal end of the sleeve device within the jejunum.

21. The method of claim 1, wherein positioning the distal end of the sleeve device within the intestine comprises positioning the distal end of the sleeve device within the duodenum.

22. The method of claim 1, wherein positioning the sleeve device occurs in the same operative session as the bariatric surgical procedure.

23. A method of treating a patient, comprising the steps of:
identifying a patient who has undergone a bariatric surgical procedure; and
positioning a gastrointestinal sleeve device with a proximal end, distal end, and elongate body in a patient such that the proximal end of the sleeve device is positioned in the stomach and the distal end of the sleeve device is positioned in the intestine.

24. A method of treating a patient, comprising the steps of:
identifying a patient who has undergone a bariatric surgical procedure; and
positioning a gastrointestinal sleeve device in a patient such that the proximal end of the sleeve device is positioned at the gastroesophageal junction and the distal end of the sleeve device is positioned in the intestine.

25. A method of treating a patient, comprising the steps of:
identifying a patient who has undergone a bariatric surgical procedure; and
positioning a gastrointestinal sleeve device in a patient such that the proximal end of the sleeve device is positioned at the gastroesophageal junction and the distal end of the sleeve device is positioned in the stomach to create a restrictive effect.

26. A method of treating a patient, comprising the steps of:
providing a gastrointestinal sleeve device, the sleeve device comprising one or more fins configured to radially expand from the sleeve device against the greater curvature of the stomach;
positioning the gastrointestinal sleeve device in the patient such that the fins radially expand against the greater curvature of the stomach; and
stapling the stomach to perform a sleeve gastrectomy such that the fins are captured in the resulting staple line, securing the fins within the neo-stomach created by the sleeve gastrectomy.
